(19) United States
(12) Patent Application Publication
(10) Pub. No.: US 2023/0084622 A1
(43) Pub. Date: Mar. 16, 2023

(54) MEDICAL INFORMATION PROCESSING APPARATUS, MEDICAL INFORMATION PROCESSING METHOD, AND MEDIUM

(71) Applicant: CANON KABUSHIKI KAISHA, Tokyo (JP)

(72) Inventor: Manami Hikosaka, Kanagawa (JP)

(21) Appl. No.: 17/941,066

(22) Filed: Sep. 9, 2022

(30) Foreign Application Priority Data
Sep. 15, 2021 (JP) 2021-150082

(51) Int. Cl. G16H 30/20; G16H 30/40

(52) U.S. Cl. CPC G16H 30/20; G16H 30/40

(57) ABSTRACT

Disclosed is a medical information processing apparatus for smoothly and appropriately grouping a plurality of medical images. The apparatus comprises: a setting acquisition unit which acquires grouping settings indicating grouping conditions for a plurality of radiation images obtained by imaging an object; and a grouping processing unit which performs a grouping of the plurality of radiation images by imparting series information identifying groups to each of the plurality of radiation images in accordance with the acquired grouping settings.

### BACKGROUND OF THE INVENTION

### Field of the Invention

The present disclosure relates to a medical information processing apparatus, a medical information processing method, and a non-transitory tangible medium

### Description of the Related Art

Conventionally, medical images imaged by medical imaging apparatuses (modalities) such as CT apparatuses and radiation imaging apparatuses are grouped into groups such as patients, modalities, inspection dates and times, and types of inspections. When a plurality of medical images are displayed on the medical image reference device, the medical images belonging to the common group are displayed side by side.

When medical image data of the DICOM standard known as a representative standard of medical image data is created, there is a function of adding series information for grouping medical images into a group called a series to the medical image. Some medical image reference apparatuses that refer to medical images have a function of displaying a plurality of medical images in a series side by side based on the series information when referring to medical images to which the series information is added.

Japanese Patent Application Laid-Open No. 2012-66119 discloses a technique for displaying on a monitor a plurality of medical images grouped according to the series information created by the modality, and correcting the series information according to the instruction when a grouping instruction different from the grouping according to the series information created by the modality is received for the plurality of medical images displayed on the monitor.

However, according to the technique disclosed in Japanese Patent Application Laid-Open No. 2012-66119, there is a point to be improved when the medical image outputted from the modality is reclassified into groups by the medical image reference apparatus.

### SUMMARY

It is therefore an object of the present disclosure to smoothly and appropriately group a plurality of medical images.

It is an object of the present disclosure to solve the above problems. Specifically, when a medical image outputted from the modality is reclassified into groups by the medical image reference device, it is necessary for a viewer to confirm the medical image and information related to the medical image, and such confirmation takes great deal of time. Further, in the conventional medical image reference apparatus in which group reclassification cannot be performed, there are cases in which medical images are displayed in unintended groups, and improvement has been required.

For dealing with such circumstance, the medical information processing apparatus according to the present disclosure comprises, an acquisition unit configured to acquire setting information indicating grouping conditions for a plurality of medical images obtained by imaging an object; and a grouping unit configured to perform a grouping of the plurality of medical images by imparting identification information for identifying a group to each of the plurality of medical images in accordance with the acquired setting information.

Further features of the present disclosure will become apparent from the following description of exemplary embodiments with reference to the attached drawings.

### BRIEF DESCRIPTION OF THE DRAWINGS

FIG. 1 is a diagram showing an example of the overall configuration of the radiation inspection system.

FIG. 2 shows an example of a grouping setting screen.

FIG. 3 shows an example of a new inspection input screen.

FIG. 4 is a view showing an example of an imaging information input screen.

FIG. 5 is a view showing an example of an imaging screen.

FIGS. 6A and 6B are flowcharts illustrating a grouping process.

FIGS. 7A, 7B and 7C are flowcharts showing details of processing for generating series information.

FIGS. 8A, 8B, 8C and 8D illustrate specific examples of grouping.

FIG. 9 is a diagram for explaining an operation for changing series information.

### DESCRIPTION OF THE EMBODIMENTS

Hereinafter, a detailed description will be given based on embodiments of the present disclosure with reference to the accompanying drawings. The configuration shown in the following embodiments is only an example, and the present disclosure is not limited to the configuration shown in the drawings.

### First Embodiment

[Configuration of Radiation Inspection System]

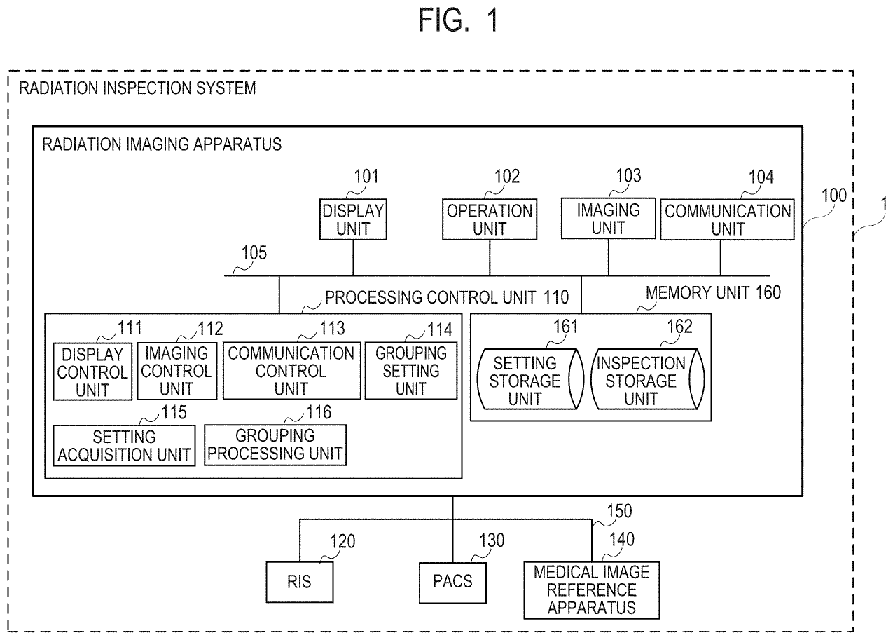

FIG. 1 shows an overall configuration example of a radiation inspection system according to this embodiment. The radiation inspection system 1 is a system for performing inspection by a radiation imaging, and includes a radiation imaging apparatus 100, an RIS 120, a PACS 130, and a medical image reference apparatus 140, according to the present embodiment. These apparatuses are connected by a network 150 and can communicate with each other.

The RIS (Radiology Information System) 120 is a so-called radiation departmental information system.

The radiation imaging apparatus 100 performs the radiation imaging of an object in accordance with an inspection order received from the RIS 120, associates information (ancillary information) relating to the radiation image with the radiation image obtained by the radiation imaging, and generates medical image data. Thereafter, the radiation imaging apparatus 100 outputs inspection information including the generated medical image data to the PACS 130.

In this embodiment, the radiation imaging apparatus 100 generates a medical image data in a file format of a data structure based on the DICOM (Digital Imaging and Communications in Medicine) standard. The radiation imaging apparatus 100 imparts series information conforming to the DICOM standard when generating the medical image data. The series information is identification information of a group called a series, and medical images can be grouped based on the series information.

The PACS (Picture Archiving and Communication Systems) 130 is an image server which is a storage device for storing the inspection information.

The medical image reference apparatus 140 is a device for displaying the medical image included in the inspection information stored in the PACS 130, and the doctor or the image reader performs a necessary diagnosis by referring to the display result. The medical image reference apparatus 140 has a function of arranging and displaying the medical images for each of series side by side on the basis of the series information when displaying the medical images to which the series information is given.

The network 150 is composed of a LAN (Local Area Network), a WAN (Wide Area Network), or the like, and may be a wired network or a wireless network.

In the present embodiment, a description will be given of an example in which a radiation image obtained by imaging an object using a radiation such as X-rays is applied as a medical image, but the present disclosure is not limited to this and other medical images may be applied. The radiation imaging apparatus 100 is an example of a medical information processing apparatus that generates series information and imparts to the radiation image obtained by imaging the object.

[Configuration of Radiation Imaging Apparatus]

Next, the details of the radiation imaging apparatus 100 will be described with reference to FIG. 1. The radiation imaging apparatus 100 includes a display unit 101, an operation unit 102, an imaging unit 103, a communication unit 104, a processing/control unit 110, and a memory unit 160, which are connected to each other by a bus 105.

The display unit 101 is composed of a liquid crystal display or the like. The display unit 101 displays various kinds of images and various kinds of information under the control of a display control unit 111 to be described later. The display contents of the display unit 101 are presented to an operator (for example, radiographers and physicians).

The operation unit 102 outputs the information input by the operator to the processing/control unit 110. The operation unit 102 includes a mouse, an operation button, and the like. The display unit 101 and the operation unit 102 may be constituted by a touch panel display in which these functions are integrated.

The imaging unit 103 is operated under the control of an imaging control unit 112, which will be described later, to image the object using the radiation.

The communication unit 104 is an interface for connecting to and communicating with a network 150.

The processing/control unit 110 is composed of a control device such as a CPU, which controls the all of the radiation imaging apparatus 100 and functions as a main part executing various processes to be described later performed by the radiation imaging apparatus 100. The processing/control unit 110 has functions of a display control unit 111, an imaging control unit 112, a communication control unit 113, a grouping setting unit 114, a setting acquisition unit 115, and a grouping processing unit 116. Details of each functional part will be described later.

The memory unit 160 is composed of a storage device such as a ROM or an HDD. The memory unit 160 stores a program. When the processing/control unit 110 executes the program stored in the memory unit 160, the processing/control unit 110 functions as each functional unit shown in FIG. 1, and the processing of a flowchart to be described later is realized.

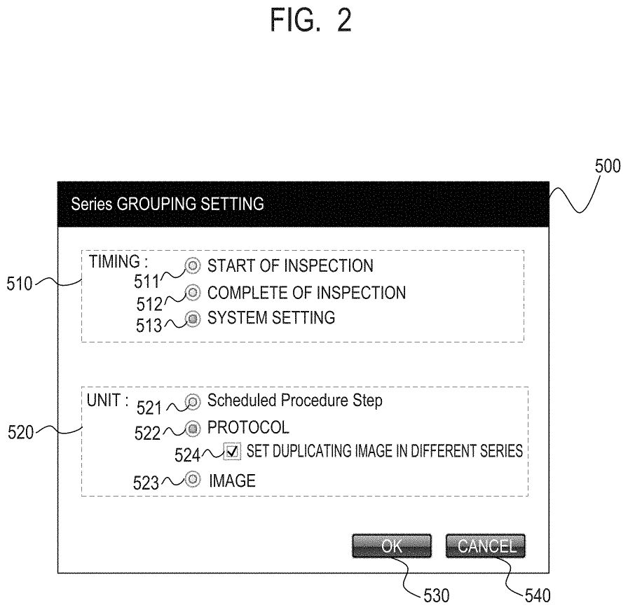

The memory unit 160 secures a storage area for storing a setting storage unit 161 and an inspection storage unit 162. The setting storage unit 161 stores setting information set by using a grouping setting screen (FIG. 2) described later. The setting information relates to conditions for grouping medical images. The inspection storage unit 162 stores the radiation image acquired from the imaging unit 103 in a file format based on the DICOM standard in association with information (ancillary information) related to the radiation image. In this embodiment, the memory unit 160 is an example of a storage unit.

Next, an operation of each functional part of the processing/control unit 110 will be described.

The display control unit 111 controls the display content of the display unit 101 based on the information input via the operation unit 102.

The imaging control unit 112 integrally controls radiation imaging of the object. The imaging control unit 112 instructs a start of the execution of the radiation imaging corresponding to at least one of the inspection orders received from the RIS 120, and operates the imaging unit 103.

The communication control unit 113 transmits and receives data to and from an external device connected to the network 150 via the communication unit 104. Specifically, the communication control unit 113 receives the inspection order from the RIS 120. The communication control unit 113 also transmits the inspection information including the medical image data generated by the processing/control unit 110 to the PACS 130.

The grouping setting unit 114 sets conditions of grouping by series information. More specifically, the setting storage unit 161 stores the setting information corresponding to the operation contents for the grouping setting screen (FIG. 2). In this embodiment, the grouping setting unit 114 functions as setting unit.

The setting acquisition unit 115 acquires the setting information from the setting storage unit 161.

When outputting the inspection information, the grouping processing unit 116 groups the plurality of radiation images to be output by applying series information to each of the plurality of radiation images to be output in accordance with the setting information acquired by the setting acquisition unit 115.

[Condition Setting of Grouping]

Next, a description will be given of a method of setting conditions for grouping by the series information in the radiation imaging apparatus 100. The radiation imaging apparatus 100 causes the display unit 101 to display the grouping setting screen for setting conditions of grouping under the control of the display control unit 111.

The grouping setting screen is displayed, for example, as one of the system setting screens of the radiation imaging apparatus 100.

FIG. 2 shows an example of the grouping setting screen 500. The grouping setting screen 500 is provided with a timing setting section 510 for setting the timing of grouping condition setting, a condition setting section 520 for setting the grouping condition, a confirm button 530, and a cancel button 540.

The operator operates radio buttons 511-513 disposed in the timing setting section 510 via the operation unit 102 to select any one of inspection start time, inspection complete time, and system setting time.

When “start of inspection” is selected by the operation of the radio button 511, the operation in the condition setting section 520 becomes invalid. Details of the processing to be executed when “start of inspection” is selected will be described later in the fourth embodiment.

When “complete of inspection” is selected by the operation of the radio button 512, the operation in the condition setting section 520 becomes invalid. The details of the processing to be executed when “complete of inspection” is selected will be described later in the second embodiment.

When “system setting” is selected by the operation of the radio button 513, the grouping setting unit 114 stores setting information corresponding to the operation contents in the condition setting section 520 in the setting storage unit 161. In the present embodiment, the following description is made assuming that “system setting” is selected.

In the condition setting section 520, radio buttons 521-523 are arranged for each of the conditions for grouping which is settable. When “system setting” is selected, the operator operates the radio buttons 521-523 via the operation unit 102 to select one of the grouping conditions.

When “Scheduled Procedure Step (hereinafter referred to as SPS)” is selected by the operation of the radio button 521, grouping is set in accordance with SPS. In the present embodiment, the SPS is information indicating a type of imaging apparatus (modality), and includes, for example, a digital X-ray apparatus (DR), a computer radiography (CR), and an angiography X-ray diagnostic apparatus (XA). In this case, the grouping processing unit 116 generates different series information for each of SPS, and imparts the same series information to the radiation images generated by the same SPS.

When “protocol” is selected by the operation of the radio button 522, grouping is set in accordance with a protocol. In this embodiment, the protocol is a previously determined imaging condition or previously determined image processing condition, used for radiation imaging, and is an imaging protocol composed of a combination of information such as an imaging position, an imaging apparatus, and a patient direction. In this case, the grouping processing unit 116 generates different series information for each of the protocols, and imparts the same series information to the radiation images generated by the same protocol.

When “protocol” is selected by the operation of the radio button 522, the ON/OFF operation of the check box 524 becomes possible. When the check box 524 is turned on, it is set that a duplicate image obtained by duplicating the radiation image obtained by the imaging unit 103 in order to perform additional image processing or the like is made into a group different from an original image which is used for obtaining the duplicating image. When the check box 524 is turned OFF, since the duplicate image is generated by the same protocol as the original image, it is set to be in the same group as the original image.

When “image” is selected by the operation of the radio button 523, it is set that each of the images is grouped individually. In this case, the grouping processing unit 116 generates different series information for each of the radiation images and imparts the generated series information to each of the radiation images.

As described above, the operator selects one of the setting for grouping in accordance with the SPS, the setting for grouping in accordance with the protocol, and the setting for grouping in accordance with the image, as a condition for grouping. Other settings may be selectable.

When the operator depresses the confirm button 530 via the operation unit 102 after the operator has completed the necessary setting, the grouping setting unit 114 stores the grouping condition selected via the condition setting section 520, as a grouping setting in the setting storage unit 161. When the operator depresses the cancel button 540 via the operation unit 102, the contents set on the grouping setting screen 500 are discarded, and the display of the grouping setting screen 500 ends. In this embodiment, series information is generated and imparted in accordance with the grouping settings stored in the setting storage unit 161.

[Flow of Inspection]

Next, a flow of an inspection using the radiation inspection system 1 shown in FIG. 1 will be described.

At the first, patient information and inspection request information are input to the radiation imaging apparatus 100 by based on an inspection request form or an inspection order from the RIS 120. The patient information includes information such as a patient name and a patient ID, and the inspection request information includes imaging information defining the content of imaging to be performed on the patient.

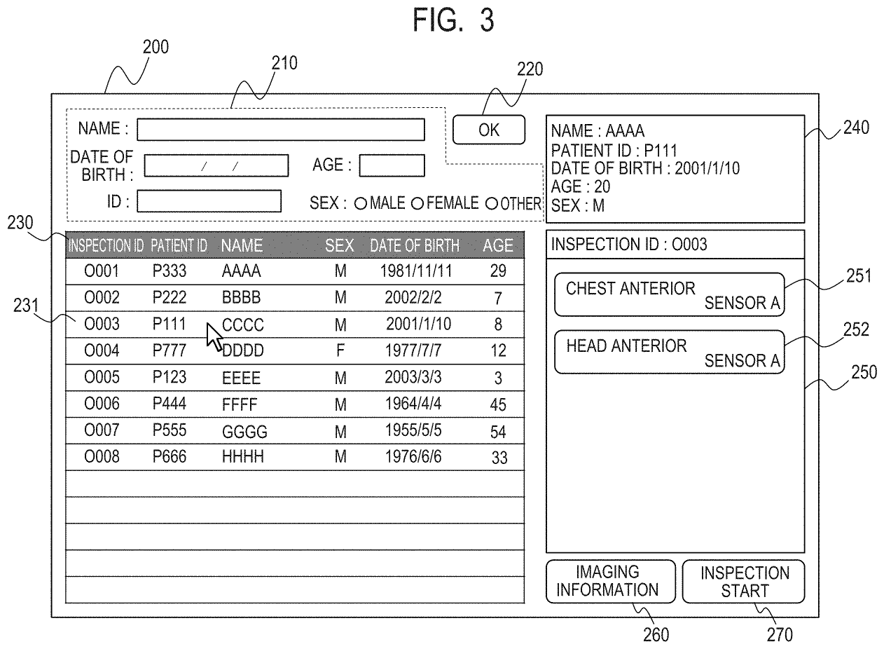

Subsequently, the radiation imaging apparatus 100 displays a new inspection input screen on the display unit 101 under the control of the display control unit 111. FIG. 3 is a diagram showing an example of a new inspection input screen 200. The new inspection input screen 200 shown in FIG. 3 includes a patient information input section 210, a patient information confirmation button 220, a requested inspection list (inspection order list) display section 230, a patient information display section 240, and an imaging information display section 250.

In the requested inspection list display section 230, an inspection orders received from the RIS 120 are arranged and displayed in a list. The inspection order received from the RIS 120 includes, for example, information about an object who is a patient, imaging information about the object including one or a plurality of imaging sites, and information (SPS) for designating a type of imaging apparatus used for imaging. Subsequently, for example, the operator selects one of the inspections 231 from the requested inspection list displayed in the requested inspection list display section 230 via the operation unit 102. By this operation, the patient information (patient ID, patient name, date of birth, etc.) corresponding to the patient of the selected inspection 231 is displayed in the patient information display section 240. The inspection ID of the selected inspection 231 is displayed in the imaging information display section 250, and the imaging information corresponding to the inspection ID is displayed in an area immediately below the area where the inspection ID is displayed. This imaging information is received from the RIS 120 as described above. In the example shown in FIG. 3, a chest anterior button 251 and a head anterior button 252, which are imaging method buttons corresponding to the imaging information, are arranged.

The new inspection input screen 200 shown in FIG. 3 is provided with an imaging information input button 260 and an inspection start button 270. For example, when the operator presses the imaging information input button 260 shown in FIG. 3 via the operation unit 102, the radiation imaging apparatus 100 displays an imaging information input screen on the display unit 101.

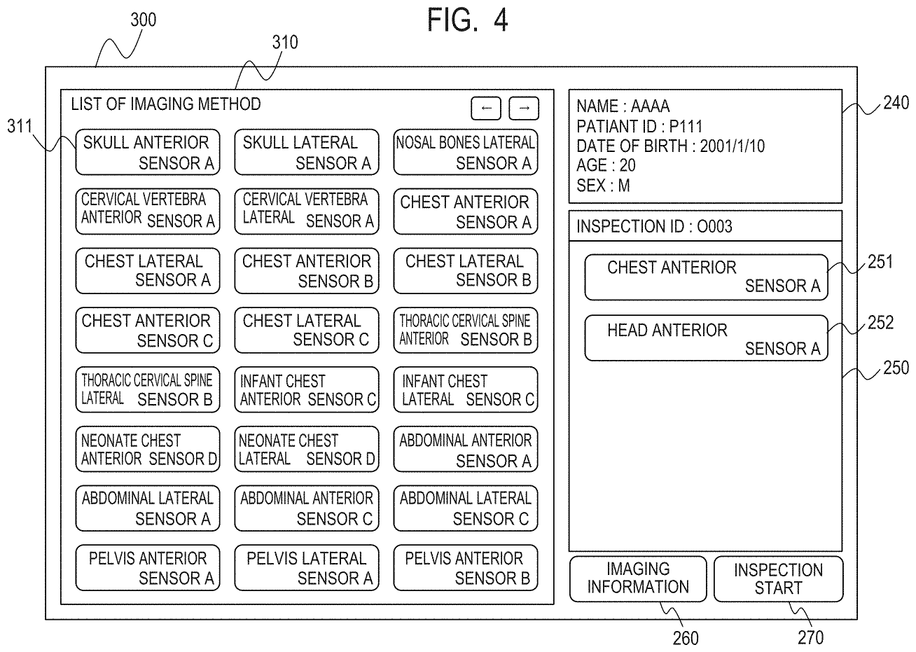

FIG. 4 is a diagram showing an example of the imaging information input screen 300. In FIG. 4, elements similar to those shown in FIG. 3 are denoted by the same reference numerals, and a detailed description thereof will be omitted.

The imaging information input screen 300 shown in FIG. 4 has a configuration in which the imaging information input section 310 is arranged in an area for displaying the patient information input section 210, the patient information confirmation button 220, and the requested inspection list display section 230 of the new inspection input screen 200 shown in FIG. 3. In the example of the imaging information input screen 300 shown in FIG. 4, a plurality of imaging method selection buttons 311 are displayed in the imaging information input section 310, and an imaging method can be added by selecting them. The added imaging method is displayed in the imaging information display section 250 side by side with the chest anterior button 251 and the head anterior button 252. Each of the imaging method is associated with an imaging method ID. The imaging method corresponds to the protocol of this embodiment.

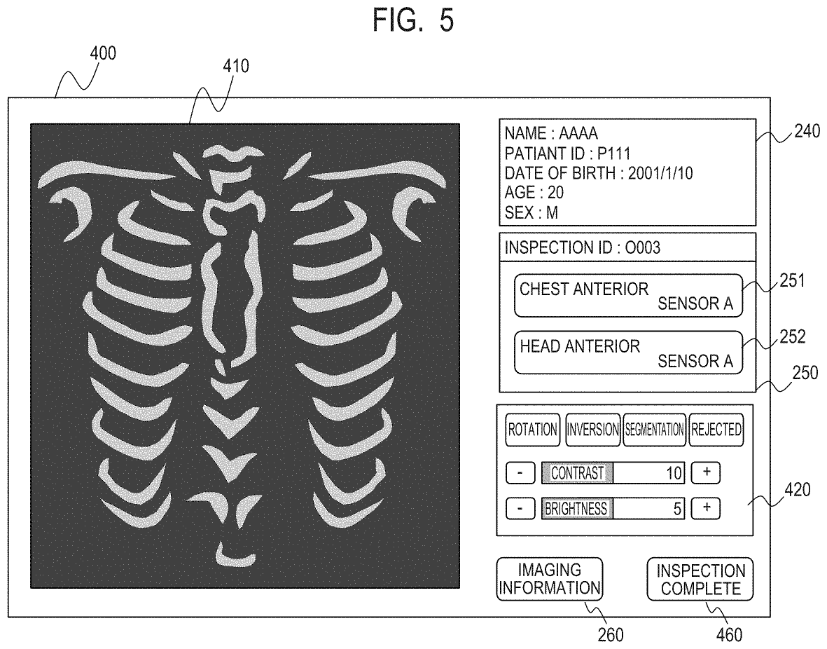

When the operator depresses the inspection start button 270 via the operation unit 102 after confirming the patient information and the imaging information displayed on the display unit 101, for example, the radiation imaging apparatus 100 determines the inspection to be performed. Then, the radiation imaging apparatus 100 displays the imaging screen on the display unit 101 in accordance with the depression of the inspection start button 270. FIG. 5 is a diagram showing an example of the imaging screen 400. The imaging screen 400 shown in FIG. 5 is a screen used for imaging, and the same reference numerals are given to the same elements as that shown in FIG. 3.

The imaging screen 400 shown in FIG. 5 is provided with an image display section 410, an image processing operation section 420, a patient information display section 240, an imaging information display section 250, an imaging information input button 260, and an inspection complete button 460.

When the imaging screen 400 shown in FIG. 5 is displayed on the display unit 101, the chest anterior button 251, which is the imaging method button disposed at the uppermost position in the imaging information display section 250, is selected by default. Accordingly, the imaging control unit 112 controls the imaging unit 103 in accordance with the imaging condition set corresponding to the imaging method button (imaging method) and the SPS included in the inspection order to prepare for radiation imaging.

Subsequently, after the operator confirms the imaging method displayed on the display unit 101, the operator performs setting of the imaging and the positioning of the patient to complete a series of the imaging preparations. Next, when the operator performs an operation for instructing the start of the radiation imaging from the operation unit 102, the imaging control unit 112 detects such operation. The imaging control unit 112 causes a radiation generation device (not shown) to irradiate the object with radiation, which is one of the configurations of the imaging unit 103, and causes a radiation detecting device (not shown) which is one of the configurations of the imaging unit 103 to detect the radiation that has passed through the object. As described above, the radiation image of the object is taken. When the imaging is completed, the imaging control unit 112 acquires a radiation image (radiation image signal) from the imaging unit 103. The imaging control unit 112 can also perform combined imaging in order to obtain a tomosynthesis image or a long image of imaging a wide area of the whole vertebral column or all lower limbs. When the combined imaging is performed, a plurality of radiation images are acquired from the imaging unit 103.

The imaging control unit 112 performs an image processing on the acquired radiation image based on predetermined image processing conditions. The predetermined image processing conditions are predetermined corresponding to, for example, the imaging method. When the image processing is completed, the radiation imaging apparatus 100 displays the image-processed radiation image on the image display section 410 and stores it in the inspection storage unit 162 in association with the imaging method (protocol) and the SPS.

When the combined imaging is made as described above, the imaging control unit 112 combines a plurality of acquired radiation images to generate a combined image. The radiation imaging apparatus 100 displays the combined image in the image display section 410, and stores each of images used for obtaining the combined image as an original image and the combined image in an inspection storage unit 162 as an image generated by the combined imaging. The same imaging method (protocol) is associated with each of the images generated by the combined imaging.

When the operator wishes to change image processing such as rotation, inversion, segmentation, contrast adjustment, brightness adjustment, etc. of the radiation image displayed in the image display section 410, the operator operates a button provided in the image processing operation section 420. In response to the operation of the button, the imaging control unit 112 performs additional image processing on the radiation image displayed in the image display section 410. The radiation imaging apparatus 100 displays the radiation image to which the additional image processing is performed in the image display section 410 and stores the radiation image as a duplicate image in an inspection storage unit 162. The same imaging method (protocol) as the original images used for the duplicate image is associated with the duplicate image.

The operator repeats the above procedure to image relating to all the imaging methods in the imaging information display section 250. When all the imaging is completed, the operator presses the inspection complete button 460. As a result, the series of the inspection is completed, and the radiation imaging apparatus 100 again displays the new inspection input screen 200 shown in FIG. 3 on the display unit 101. The processing/control unit 110 generates medical image data by associating inspection contents, imaging conditions, series information, and the like as ancillary information with the images not handled as a rejected image among the radiation images stored in the inspection storage unit 162.

The communication control unit 113 controls the communication unit 104 to transmit the inspection information including the generated medical image data to a storage device such as the PACS 130.

[Grouping Processing]

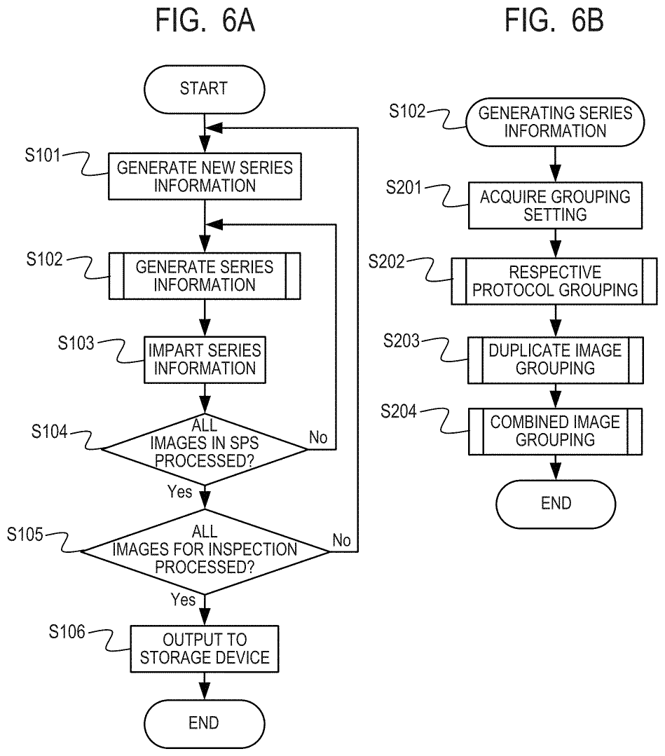

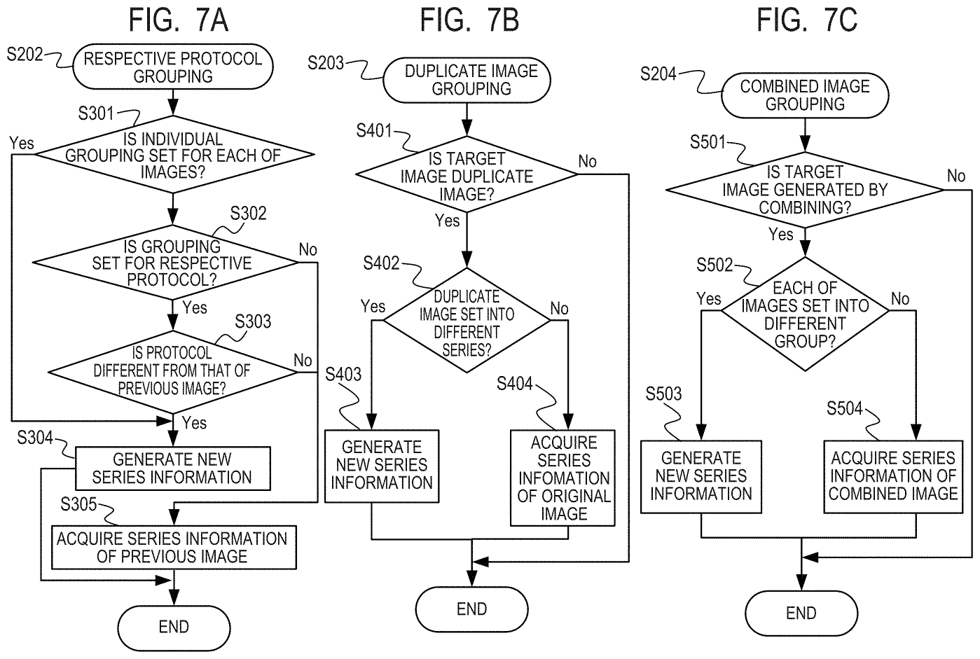

Next, with reference to FIGS. 6A to 8D, grouping processing executed by the radiation imaging apparatus 100 according to the present embodiment will be described. FIG. 6A is a flowchart showing an example of grouping processing. The process of the flowchart of FIG. 6A is a process started in response to an instruction to output the inspection information, and is started, for example, when the inspection complete button 460 of the imaging screen 400 of FIG. 5 is depressed. Note that the inspection output button may be separately provided between, for example, the imaging information input button 260 and the inspection complete button 460 on the imaging screen 400 of FIG. 5, and the process of the flowchart of FIG. 6A may be started when the inspection output button is depressed. The flowcharts shown in FIGS. 6A to 7C are implemented by the processing/control unit 110 executing a program stored in the memory unit 160. Hereinafter, each of steps (step) in the flowchart will be described by giving an S (step) at the head of the reference numerals.

In the present embodiment, it is assumed that, before the flowchart of FIG. 6A is started, a process of associating information related to the radiation image (inspection contents, imaging conditions, etc. other than the series information) with the radiation image stored in the inspection storage unit 162 is performed. Note that such associating processing may be performed at any timing in the flowchart of FIG. 6A before the execution of the output processing (S106) to the storage device.

Firstly, in step S101, the grouping processing unit 116 selects one SPS in the inspection to be output and makes it a target. Then, a first image in the radiation images belonging to the SPS is set as a target image, among the radiation images stored in the inspection storage unit 162, and new series information for imparting to the target image is generated. Subsequently, the process skips step S102 and proceeds to step S103. In this case, in step S103, the grouping processing unit 116 imparts the series information generated in step S101 to the target image. Thereafter, in step S104, the grouping processing unit 116 sets the next image among the radiation images belonging to the SPS as the target image, and the processing proceeds to step S102.

In step S102, the grouping processing unit 116 performs processing for generating series information. The process of generating the series information will be described with reference to FIG. 6B.

FIG. 6B is a flowchart showing details of the processing for generating the series information, which is executed in step S102 of FIG. 6A.

At step S201, the setting acquisition unit 115 acquires the grouping setting from the setting storage unit 161. The timing for acquiring the grouping setting may be the start of the radiation imaging apparatus 100 or the start of the processing of the flowchart shown in FIG. 6A. In this embodiment, the setting acquisition unit 115 functions as an acquisition unit.

Subsequently, the grouping processing unit 116 executes the respective protocol grouping processing (step S202), the duplicate image grouping processing (step S203), and the combined image grouping processing (step S204) in accordance with the grouping settings acquired in step S201. Thereafter, the process proceeds to step S103 in FIG. 6A. The details of the processes in step S202-S204 will be described with reference to FIGS. 7A to 7C.

Firstly, the respective protocol grouping process (step S202) will be described. FIG. 7A is a flowchart showing details of the respective protocol grouping process step (step S202).

In step S301, the grouping processing unit 116 determines whether or not an individual group has been set for each of images based on the grouping setting acquired in step S201. If it is determined that the images are grouped individually, the process proceeds to step S304, otherwise, the process proceeds to step S302.

In step S302, the grouping processing unit 116 determines whether grouping has been set for each of the protocols based on the grouping setting acquired in step S201. If grouping is set for each of the protocols, the process proceeds to step S303, otherwise, is it determined that the grouping is set for each SPS, the process proceeds to step S305.

In step S303, the grouping processing unit 116 determines whether or not the protocol of the target image is different from the protocol of the image (previous image) as the last target image. If the protocol of the target image is different from that of the previous image (for example, when multiple protocols are used for a single inspection), the process proceeds to step S304. If the protocol of the target image is the same as that of the previous image (for example, in the case where a plurality of times of imaging is performed with one protocol for generating the combined image), the process proceeds to step S305.

In step S304, the grouping processing unit 116 generates new series information. As a result, when it is set that the grouping is performed for each of the protocols, different series information is generated for each of the protocols. In addition, when setting is made to individual groups for each of the images, different series information is generated for each of the images. Thereafter, the process proceeds to step S203.

In step S305, the grouping processing unit 116 acquires the series information of the previous image. As a result, when it is set that the grouping is performed for each of the protocols, the series information of images having the same protocol is acquired. When it is set that the grouping is performed for each of the SPS, the series information of the first image in the target SPS is acquired. Thereafter, the process proceeds to step S203.

The radiation imaging apparatus 100 sequentially repeats the above-described grouping processing for respective protocol with respect to the radiation image belonging to the SPS. By executing such process, it becomes possible to perform grouping for each of the protocols or to perform grouping for each of the imaging in accordance with the setting.

Next, the duplicate image grouping process (step S203) will be described. FIG. 7B is a flowchart showing details of the duplicate image grouping process (step S203). The process of the flowchart shown in FIG. 7B is a process executed when grouping is set for each of the protocols.

In step S401, the grouping processing unit 116 determines whether or not the target image is a duplicate image. If the image is a duplicate image, the process proceeds to step S402, and if the image is not the duplicate image, the subsequent steps are skipped, and the process proceeds to step S204.

In step 402, the grouping processing unit 116 determines whether or not the setting for grouping the duplicate image and the original image used for obtaining the duplicate image into different groups is enable. If the setting for grouping the duplicate image and the original image into different groups is enabled, the process proceeds to step S403, otherwise, the process proceeds to step S404.

In step S403, the grouping processing unit 116 generates new series information. As a result, series information different from that of the original image is generated for the duplicate image. Thereafter, the process proceeds to step S204.

In step S404, the grouping processing unit 116 acquires the series information of the original image.

If the series information of the original image has already been acquired by the processing in step S305, this step may be skipped. Thereafter, the process proceeds to step S204.

According to the above-described duplicate image grouping processing, it is possible to switch between the duplicate image and the original image being set in different groups or in the same group in accordance with the setting when grouping is performed for respective protocol.

Next, the combined image grouping process (step S204) will be described. FIG. 7C is a flowchart showing details of the combined image grouping process (step S204). In this flowchart, series information for imparting to an image (each of the images used for obtaining a combined image and the combined image) generated by combined imaging is generated.

In step S501, the grouping processing unit 116 determines whether or not the target image is an image generated by combining original images. If the target image is generated by combining the original images, the process proceeds to step S502, and if the target image is not generated by combining the original images, the process skips subsequent steps and proceeds to step S103 in FIG. 6A.

In step S502, the grouping processing unit 116 determines whether or not each of images are set into individual group based on the grouping setting acquired in step S201. If it is determined that the images are grouped individually, the process proceeds to step S503, otherwise, the process proceeds to step S504.

In step S503, the grouping processing unit 116 generates new series information. As a result, different series information are generated for each of the original images and the combined image. If the series information for each of the images has already been generated by the processing in step S304, this step may be skipped. Thereafter, the process proceeds to step S103 in FIG. 6A.

In step S504, the grouping processing unit 116 acquires the series information of the combined image. Specifically, when the target image is the first image of the original images used for obtaining the combined image, new series information is generated, and when the target image is the second image or later of the original images, series information of the first image in the same combined imaging is acquired. When the target image is the combined image, series information of the original images is acquired. Thereafter, the process proceeds to step S103 in FIG. 6A.

By the combined image grouping processing as described above, it is possible to switch whether a series of image groups (combined image groups) generated by the combined imaging is made into individual groups or the same group for each image in accordance with the setting.

Next, the description returns to FIG. 6A.

In step S103, the grouping processing unit 116 imparts the series information generated or acquired in the process of generating the series information (step 102) to the target image. In this embodiment, the grouping processing unit 116 functions as a grouping unit.

Next, in step S104, the grouping processing unit 116 sequentially repeats a process of generating series information (step S102) and a process of imparting series information (step S103) to the image belonging to the target SPS. When the processes of step S102 and step S103 are completed for all the images belonging to the target SPS, the process proceeds to step S105.

In step S105, the grouping processing unit 116 determines whether the processing in steps S102 and S103 has been completed for all the images in the inspection of the output target. If there is an image that has not been processed in steps S102 and S103 in the inspection of the output target, the processing proceeds to step S101 with one of the unprocessed SPSs in the inspection as the output target, and the grouping processing unit 116 generates new series information. Thus, different series information is generated for each of the SPSs. If grouping is set for each of the SPSs, in the processes of steps S102 and S103, the series information generated in step S101 is acquired and imparted to all images belonging to the target SPSs. That is, the SPSs can be grouped in accordance with the setting. When the processing of steps S102 and S103 is completed for all images in the inspection of the output target, the processing proceeds to step S106.

At step S106, the communication control unit 113 transmits to the PACS 130 all images in the inspection to which the ancillary information including the series information is imparted. In this embodiment, the processing of steps S101-S105 are executed for all the images in the inspection, and after all the images are grouped, the communication control unit 113 controls to output inspection information including all the images to the PACS 130 as a storage device. In this embodiment, the communication control unit 113 functions as the output control unit. Thereafter, the processing of the series of flowcharts completes.

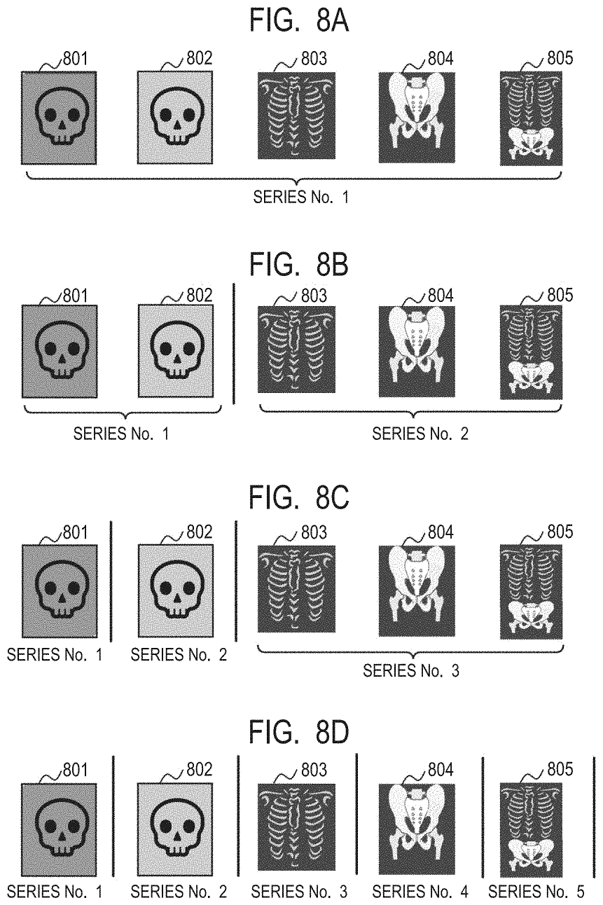

FIGS. 8A to 8D are diagrams for explaining a specific example of grouping performed according to the grouping setting. FIGS. 8A to 8D describe a case where the images 801-805 are grouped. The image 801 is an acquired image of the anterior of the head. The image 802 is a duplicate image of the image 801. The image 803 is the firstly acquired image obtained by the combined imaging. The image 804 is the secondly acquired image obtained by the combined imaging. The image 805 is a combined image of the image 803 and the image 804. The images 801-805 are generated by the same SPS. The images 803-805 are generated by a protocol different from that of the images 801-802. In FIGS. 8A to 8D, the grouping process shown in FIGS. 6A and 7C is performed on the images 801-805, and a series number is assigned as an example of series information.

FIG. 8A shows the result of grouping in the case where grouping is set for each of the SPSs. The images 801-805 are imparted a series number “1”. In this setting, the images 801-805 are all classified into the same group.

FIG. 8B is a diagram showing the result of grouping in the case where grouping is set for each of the protocols. The series number “1” is imparted to the images 801-802, and the series number “2” is imparted to the images 803-805. In this configuration, images 801-805 are grouped in accordance with the protocol.

FIG. 8C is a diagram showing the result of grouping in the case where grouping is set for each of the protocols and the setting for grouping the duplicate image and the original image used for generating the duplicate image, into different groups is enabled. The series number “1” is imparted to the image 801, the series number “2” is imparted to the image 802, and the series number “3” is imparted to the images 803-805. In this setting, although the images are grouped in accordance with the protocol, the duplicate images are classified into a group different from the original image.

FIG. 8D is a diagram showing the result of grouping in the case where grouping is set for each of the images. Series numbers “1” to “5” are respectively imparted to the images 801-805. In this setting, the images 801-805 are classified into different groups. Therefore, the images generated by the combined imaging are also classified into different groups.

As described above, when outputting the radiation images obtained by the inspection to the storage device such as the PACS 130, the radiation imaging apparatus 100 according to the present embodiment generates and imparts series information to each of the images in accordance with the set grouping conditions, and groups the images. Thus, when the medical images stored in the PACS 130 or the like are referred to by using the medical image reference apparatus 140, by using for example, grouping conditions in accordance with the application, comparison and referring of the medical images can be made without the user of the medical image reference apparatus 140 performing the group sorting operation. In addition, even in the medical image reference apparatus 140 which cannot perform the group sorting operation, it is possible to compare and refer appropriately grouped medical images. As described above, according to the present embodiment, appropriate grouping of medical images can be smoothly performed. That is, comparison reading at the time of diagnosis by the physician is facilitated.

Although the above description exemplifies a configuration in which grouping settings are acquired from the setting storage unit 161, a configuration in which grouping settings are acquired from a storage device such as the PACS 130 may be used as the first modification of the present embodiment. In this case, for example, the radiation imaging apparatus 100 acquires grouping settings from the PACS 130 via the network 150, and performs grouping in accordance with the acquired grouping settings. In this embodiment, the PACS 130 has the setting unit for setting conditions for grouping by series information, and the memory unit for storing grouping settings set by the setting unit.

As a second modification of the present embodiment, when a plurality of PACS are connected to the network 150 of the radiation inspection system 1, the radiation imaging apparatus 100 may store different grouping settings for each of the PACSs. In this case, for example, the radiation imaging apparatus 100, under the control of the display control unit 111 while designating any of the connected PACSs, displays on the display unit 101 the grouping setting screen 500 on which the timing setting section 510 is not displayed. A grouping setting unit 114 stores the grouping setting set in the grouping setting screen 500 in the setting storage unit 161 in association with the designated PACS. Thus, the grouping setting is stored in the setting storage unit 161 for each of PACSs. In this case, for example, when outputting the inspection information, the radiation imaging apparatus 100 acquires the grouping setting in accordance with the PACS to which the inspection information is output, from the setting storage unit 161, and performs grouping in accordance with the acquired grouping setting.

### Second Embodiment

Next, a second embodiment of the present disclosure will be described. In the description of the second embodiment described below, matters common to the first embodiment described above will be omitted, and matters different from the first embodiment described above will be mainly described.

The schematic configuration of the radiation inspection system including the radiation imaging apparatus according to the second embodiment is the same as that of the radiation inspection system 1 including the radiation imaging apparatus 100 according to the first embodiment shown in FIG. 1. In the first embodiment described above, a grouping method in accordance with the set grouping conditions has been described. On the other hand, in the second embodiment, a method in which the operator confirms and changes the grouping results will be described.

In a case of setting the grouping conditions according to the present embodiment, the operator selects “complete of inspection” of the timing setting section 510 on the grouping setting screen 500 shown in FIG. 2.

In this case, when the inspection information is outputted, the grouping can be confirmed and changed. It should be noted that the grouping setting screen 500 is not provided with the timing setting section 510, and the grouping can be confirmed and changed when the inspection information is output. Since the flow of the inspection in this embodiment is the same as that in the first embodiment, the description thereof will be omitted.

Next, a grouping process executed by the radiation imaging apparatus 100 according to the present embodiment will be described. Similar to the first embodiment, this grouping process is started in response to an output instruction of the inspection information. Specifically, when the inspection complete button 460 of the imaging screen 400 of FIG. 5 is pressed, the processing/control unit 110 associates inspection contents, imaging conditions, series information, and the like as ancillary information with images which is not treated as the rejected image among the radiation images stored in the inspection storage unit 162. As a screen for displaying the result of grouping by using the series information as the ancillary information, the radiation imaging apparatus 100 displays a grouping display screen on the display unit 101 under the control of the display control unit 111. For example, the radiation imaging apparatus 100 executes the processing of steps S101-S105 in FIG. 6A in accordance with the default grouping setting, and displays the grouping display screen before executing the processing of step S106.

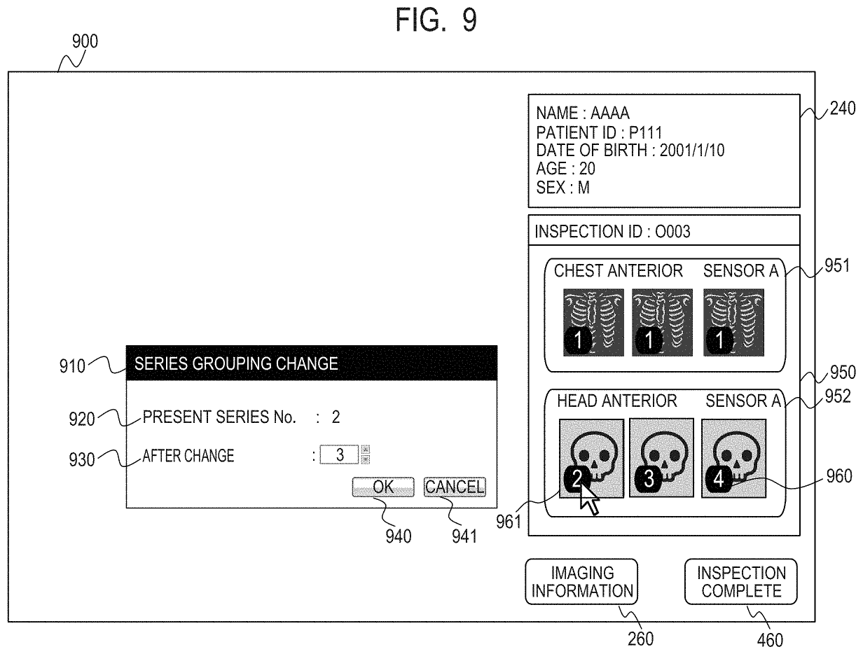

FIG. 9 shows an example of the grouping display screen 900. The grouping display screen 900 shown in FIG. 9 has the same reference numerals as those of the configuration shown in FIG. 5. The grouping display screen 900 shown in FIG. 9 is provided with a patient information display section 240, a change input window 910, an acquired image display section 950, an imaging information input button 260, and an inspection complete button 460. Note that the change input window 910 is not displayed in the initial state.

The acquired image display section 950 corresponds to the imaging information display section 250 of the imaging screen 400 shown in FIG. 5. In the acquired image display section 950, images obtained by imaging are displayed for each of the imaging method displayed in the imaging information display section 250. In the example shown in FIG. 9, a chest anterior image display section 951 for displaying an image group generated corresponding to the chest anterior button 251 and a head anterior image display section 952 for displaying an image group generated corresponding to the head anterior button 252 are displayed side by side in the acquired image display section 950. A display item 960 representing a series number currently imparted to the image is superimposed on each image displayed in the acquired image display section 950. The series number is an example of series information. If the correspondence between each of the images obtained by imaging and the series number imparted to the image is visible, the display configuration of the acquired image display section is not limited to the display configuration shown in FIG. 9.

In the present embodiment, since grouping is set for each of the protocols in the default grouping setting, the display item 960 displays the series number generated and imparted for each of the protocols as an initial value. Note that the default grouping setting is not limited to a setting for grouping in accordance with the protocol, but may be a setting for grouping in accordance with the SPS or a setting for grouping in accordance with images.

Subsequently, for example, when the operator selects one of the display items 961 from the display items displayed on each image in the acquired image display section 950 via the operation unit 102, the change input window 910 described above is displayed on the grouping display screen 900. The change input window 910 is provided with a number display section 920, a number changing section 930, a confirm button 940, and a cancel button 941.

The number display section 920 displays the series number currently imparted to the selected image. When the grouping is changed, the operator operates a number changing section 930 via the operation unit 102 to select a series number to which the current number is changed. When the change is completed and the confirm button 940 is depressed, a grouping processing unit 116 changes the series number imparted to the selected image to the series number selected by the number changing section 930. The series number displayed on the display item 961 is updated to the changed series number. When the cancel button 941 is depressed, the display control unit 111 discards the operation contents in the change input window 910 and closes the change input window 910.

As described above, the operator checks and changes the series number imparted to each of the images in the inspection of the output target. When the inspection complete button 460 on the grouping display screen 900 of FIG. 9 is depressed, the communication control unit 113 transmits to the PACS 130 all images in the inspection to which the ancillary information including the series information is imparted.

As described above, according to this embodiment, when outputting the inspection information, the operator can confirm and change the grouping results. This makes it possible to perform a grouping suitable for each of the inspections, and makes it easy to perform comparative reading at the time of diagnosis by a physician.

In the above description, the series information is changed for each of the images when the inspection information is output, but the method of changing the series information is not limited to this, and the series information may be changed for each of the protocols or the series information may be changed for each of the SPSs.

As a modification of the present embodiment, the radiation imaging apparatus 100 may display the grouping setting screen 500 in which the timing setting section 510 is not displayed under the control of the display control unit 111 at the completion of the inspection. In this case, the grouping processing unit 116 generates and imparts series information in accordance with the setting on the grouping setting screen 500, and performs grouping. In this modification, grouping is performed in accordance with the grouping condition set at the completion of the inspection.

### Third Embodiment

Next, a third embodiment of the present disclosure will be described. In the description of the third embodiment described below, matters common to the first embodiment described above will be omitted, and matters different from the first embodiment described above will be mainly described.

The schematic configuration of the radiation inspection system including the radiation imaging apparatus according to the third embodiment is the same as that of the radiation inspection system 1 including the radiation imaging apparatus 100 according to the first embodiment shown in FIG. 1. In the first embodiment described above, the same grouping setting is applied to all images in the inspection to perform grouping. On the other hand, in the third embodiment, a method of performing grouping by applying grouping settings set for each protocol will be described.

In the present embodiment, when grouping conditions are set, each of the protocols is set using a protocol setting screen (not shown). When setting for each of the protocols is performed, the radiation imaging apparatus 10 displays a grouping setting screen 500 with the timing setting section 510 hidden on the display unit 101 under the control of the display control unit 111. The grouping setting unit 114 stores the grouping setting set in the grouping setting screen 500 in the setting storage unit 161 in association with the target protocol. Thus, the grouping setting is stored in the setting storage unit 161 for each of the protocols. Since the flow of the inspection in this embodiment is the same as that in the first embodiment, the description thereof will be omitted.

Next, a grouping process executed by the radiation imaging apparatus 100 according to the present embodiment will be described. Similar to the first embodiment, this grouping process is started in response to an output instruction of the inspection information. The difference from the first embodiment is that in step S201 of FIG. 6B, when the setting acquisition unit 115 acquires the grouping setting, it acquires the grouping setting corresponding to the protocol of the target image from the setting storage unit 161. Then, in the subsequent processing of the flowchart, processing for generating and imparting series information to the target image is executed according to the imparted grouping setting.

According to the grouping processing of the present embodiment as described above, the same series information as the images of other protocols in the same SPS is given to the images of the protocols which are set to be grouped by each of the SPSs. As in the first embodiment, series information generated for each of the protocols and series information generated for each of the images are imparted to the images of the protocols for which grouping is set for each of the protocols or to the images of the protocols for which grouping is set for each of the images

As described above, according to the present embodiment, it is possible to perform grouping suitable for each of the protocols by applying grouping conditions set for each of the protocols. Therefore, comparison reading is facilitated at the time of diagnosis by the physician.

### Fourth Embodiment

Next, a fourth embodiment of the present disclosure will be described. In the description of the fourth embodiment described below, matters common to the first embodiment described above will be omitted, and matters different from the first embodiment described above will be mainly described.

The schematic configuration of the radiation inspection system including the radiation imaging apparatus according to the fourth embodiment is the same as that of the radiation inspection system 1 including the radiation imaging apparatus 100 according to the first embodiment shown in FIG. 1. In the first embodiment described above, the method of performing grouping in accordance with the predetermined grouping condition has been described. On the other hand, in the fourth embodiment, a method of performing grouping in accordance with the grouping condition set at the start of the inspection will be described.

To set the conditions for grouping according to the present embodiment, the operator selects “at the start of inspection” of the timing setting section 510 on the grouping setting screen 500 shown in FIG. 2.

In this case, the radiation imaging apparatus 100 displays the grouping setting screen 500 in which the timing setting section 510 is not displayed on the display unit 101 under the control of the display control unit 111 when the inspection is started. A grouping setting unit 114 stores the grouping setting set in the grouping setting screen 500 in the setting storage unit 161. Note that the grouping setting screen 500 may be configured such that the timing setting section 510 is not provided and the grouping condition is set when the inspection is started. Since the flow of the inspection in this embodiment is the same as that in the first embodiment, the description thereof will be omitted.

Next, a grouping process executed by the radiation imaging apparatus 100 according to the present embodiment will be described. Similar to the first embodiment, this grouping process is started in response to an output instruction of the inspection information. The difference from the first embodiment is that in step S201 of FIG. 6B, the setting acquisition unit 115 acquires the grouping setting set at the time of starting the inspection from the setting storage unit 161. Then, in the subsequent processing of the flowchart, processing for generating and imparting series information to the target image is executed according to the acquired grouping setting.

As described above, according to the present embodiment, in accordance with the grouping condition set at the time of starting the inspection, suitable grouping can be performed for each of the inspections. That is, comparison reading is facilitated at the time of diagnosis by the physician.

Although the present disclosure has been described together with the embodiments described above, the embodiments described above are merely examples of embodiments for carrying out the present disclosure, and the technical scope of the present disclosure should not be construed in a limited manner. That is, the present disclosure can be practiced in various ways without departing from its technical philosophy or key features.

According to the present disclosure, it can be executed to smoothly and appropriately group a plurality of medical images.

### Other Embodiment

Embodiments of the present disclosure can also be realized by a computer of a system or apparatus that reads out and executes computer executable instructions recorded on a storage medium (e.g., non-transitory computer-readable storage medium) to perform the functions of one or more of the above-described embodiment(s) of the present disclosure, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuity, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2021-150082, filed Sep. 15, 2021, which is hereby incorporated by reference herein in its entirety.

What is claimed is:

1. A medical information processing apparatus comprising:
an acquisition unit configured to acquire setting information indicating grouping conditions for a plurality of medical images obtained by imaging an object; and
a grouping unit configured to perform a grouping of the plurality of medical images by imparting identification information for identifying a group to each of the plurality of medical images in accordance with the acquired setting information.

2. The medical information processing apparatus according to claim 1, wherein in a case that in the acquired setting information the grouping is performed in accordance with each of imaging protocols, the grouping unit generates the identification information different for the each of the imaging protocols, and imparts the same identification information to medical images generated by the same imaging protocol.

3. The medical information processing apparatus according to claim 2, wherein even in a case that in the acquired setting information the grouping is performed in accordance with the each of imaging protocols, when it is set that a duplicate image generated by duplicating the medical image and an original medical image which is the medical image used for generating the duplicate image are to be grouped separately, the grouping unit generates different identification information different from the identification information of the original medical image and imparts the different identification information to the duplicating image.

4. The medical information processing apparatus according to claim 1, wherein in a case that in the acquired setting information the grouping is performed in accordance with each of the plurality of medical images individually, the grouping unit generates different identification information different for the each of the plurality of medical images and imparts the different identification information to the each of the plurality of medical images.

5. The medical information processing apparatus according to claim 1, wherein the grouping unit generates the same identification information for a combined image group including a combined image generated by combining the plurality of medical images and original medical images which are included in the plurality of medical images and are used for generating the combined image, and imparts the same identification information to the images included in the combined image group.

6. The medical information processing apparatus according to claim 5, wherein in a case that in the acquired setting information the grouping is performed in accordance with each of the plurality of medical images individually, the grouping unit generates different identification information different for each of the images included in the combined image group and imparts the different identification information to each of the images included in the combined image group.

7. The medical information processing apparatus according to claim 1, wherein in a case that in the acquired setting information the grouping is performed in accordance with types of imaging apparatuses individually, the grouping unit generates the different identification information different for each type of the imaging apparatus, and imparts the same identification information to medical images generated by the imaging apparatus of the same type.

8. The medical information processing apparatus according to claim 1, further comprising a setting unit configured to set the grouping conditions.

9. The medical information processing apparatus according to claim 8, wherein the setting unit sets the grouping condition at a start or an end of imaging the object.

10. The medical information processing apparatus according to claim 1, further comprising a memory unit configured to store the setting information indicating the grouping condition,
wherein the acquisition unit acquires the setting information from the memory unit.

11. The medical information processing apparatus according to claim 10, wherein the memory unit stores the setting information for the each of imaging protocols, and the acquisition unit acquires, from the memory unit, the setting information corresponding to the imaging protocol used for generating a target medical image.

12. The medical information processing apparatus according to claim 1, further comprising an output control unit configured to control, after grouping the plurality of medical images, to output inspection information including the plurality of medical images to which the identification information is imparted to a storage device.

13. The medical information processing apparatus according to claim 12, further comprising a memory unit configured to store the setting information indicating the grouping condition for each of the storage devices to which the inspection information is output,
wherein the acquisition unit acquires the setting information corresponding to the storage device to which the inspection information is output, from the memory unit.

14. The medical information processing apparatus according to claim 12, wherein the acquiring unit acquires setting information indicating the grouping condition from the storage device to which the inspection information is output.

15. The medical information processing apparatus according to claim 12, further comprising a display control unit configured to perform a control of displaying the plurality of medical images in association with the identification information imparted to the each of the plurality of medical images after the plurality of medical images are grouped,
wherein the grouping unit changes the identification information imparted to the displayed medical image in accordance with an operation of an operator, and
after changing, the output control unit controls to output the inspection information to the storage device.

16. The medical information processing apparatus according to claim 1, wherein the identification information is series information of the DICOM standard.

17. The medical information processing apparatus according to claim 1, further comprising a radiation imaging apparatus configured to imaging a medical image by using radiation.

18. A medical information processing method comprising:
an acquisition step of acquiring setting information indicating grouping conditions for a plurality of medical images obtained by imaging an object; and
a grouping step of performing a grouping of the plurality of medical images by imparting identification information for identifying a group to each of the plurality of medical images in accordance with the acquired setting information.

19. A non-transitory tangible medium having recorded thereon a program for causing a computer to execute respective steps of the medical information processing method according to claim 18.
